(19) United States
(12) Patent Application Publication
(10) Pub. No.: US 2008/0200438 A1
(43) Pub. Date: Aug. 21, 2008

(54) Treatment of Autoimmune Diseases

(76) Inventors: Rainer Albert, Basel (CH); Nigel Graham Cooke, Oberwil (CH); Barbara Nusslein-Hildesheim, Eimeldingen (DE); Sven Weiler, Lorrach (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/065,096

(22) PCT Filed: Sep. 7, 2006
(86) PCT No.: PCT/EP2006/066150

§ 371 Date: Feb. 28, 2008

Related U.S. Application Data
(60) Provisional application No. 60/715,990, filed on Sep. 9, 2005.

(51) Int. Cl. A61K 31/661; A61P 25/00; A61K 31/137

(52) U.S. Cl. 514/114; 514/649

(57) ABSTRACT

Methods of treating various autoimmune diseases, such as multiple sclerosis, peripheral neuritis, optical neuritis, amylotrophic lateral sclerosis, and uveitis utilizing specific amino alcohol derivatives are provided herein.

### FIELD OF THE INVENTION

The present invention relates generally to amino alcohols and derivatives thereof, and more specifically to their use to treat particular autoimmune diseases, such as multiple sclerosis, peripheral neuritis, optical neuritis, amyotrophic lateral sclerosis and uveitis.

### BACKGROUND OF THE INVENTION

Multiple sclerosis is a chronic inflammatory disease of the central nervous system (CNS) with an unknown pathophysiological cause. Clinical manifestations are associated with the infiltration of the central nervous system by immune-competent cells. Specific T cell populations directed towards neuroantigens, such as myelin basic protein, can be demonstrated in the periphery. This suggests the involvement of an autoimmune response in the development of the disease. Although there is no specific treatment for this T cell-mediated autoimmune disorder, patients receive immunosuppressive therapy including azathioprine and corticosteroids in order to limit the extent of the inflammatory process. Immunosuppressive therapy of multiple sclerosis, however, is only partially effective, and in most cases only offers a delay in disease progression despite anti-inflammatory and immunosuppressive treatment.

Accordingly, there is a need for other therapeutics which are effective in the treatment of multiple sclerosis and other related diseases including those involving T-cell mediated damage to central or peripheral nerve tissue, such as peripheral neuritis, optical neuritis and amyotrophic lateral sclerosis.

It has now been found that an amino alcohol such as disclosed thereafter has a beneficial effect in the treatment of autoimmune diseases such as multiple sclerosis, peripheral neuritis, optical neuritis, amyotrophic lateral sclerosis (Lou Gehrig's disease) or uveitis.

Amino alcohols which can be used according to the invention are compounds of formula I

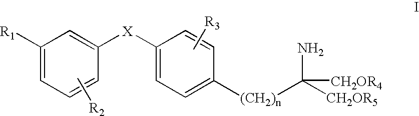

wherein X is O, S, SO or SO₂;
R₁ is halogen, trihalomethyl, OH, C₁₋₇alkyl, C₁₋₄alkoxy, trifluoromethoxy, phenoxy, cyclohexylmethyloxy, pyridylmethoxy, cinnamyloxy, naphthylmethoxy, phenoxymethyl, CH₂—OH, CH₂—CH₂—OH, C₁₋₄alkylthio, C₁₋₄alkylsulfinyl, C₁₋₄alkylsulfonyl, benzylthio, acetyl, nitro or cyano, or phenyl, phenylC₁₋₄alkyl or phenyl-C₁₋₄alkoxy each phenyl group thereof being optionally substituted by halogen, CF₃, C₁₋₄alkyl or C₁₋₄alkoxy;
R₂ is H, halogen, trihalomethyl, C₁₋₄alkoxy, C₁₋₇alkyl, phenethyl or benzyloxy;
R₃H, halogen, CF₃, OH, C₁₋₇alkyl, C₁₋₄alkoxy, benzyloxy, phenyl or C₁₋₄alkoxymethyl;
each of R₄ and R₅, independently is H or a residue of formula (a)

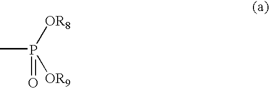

wherein each of R₈ and R₉, independently, is H or C₁₋₄alkyl optionally substituted by halogen; and
n is an integer from 1 to 4;
or a pharmaceutically acceptable salt thereof,
or a compound of formula II

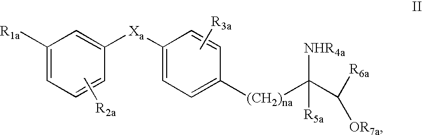

wherein
R₁ₐ is halogen, trihalomethyl, C₁₋₄alkyl, C₁₋₄alkoxy, C₁₋₄alkylthio, C₁₋₄alkylsulifinyl, C₁₋₄alkyl-sulfonyl, aralkyl, optionally substituted phenoxy or aralkyloxy;
R₂ₐ is H, halogen, trihalomethyl, C₁₋₄alkyl, C₁₋₄alkoxy, aralkyl or aralkyloxy;
R₃ₐ is H, halogen, CF₃, C₁₋₄alkyl, C₁₋₄alkoxy, C₁₋₄alkylthio or benzyloxy;
R₄ₐ is H, C₁₋₄alkyl, phenyl, optionally substituted benzyl or benzoyl, or lower aliphatic C₁₋₅acyl;
R₅ₐ is H, monohalomethyl, C₁₋₄alkyl, C₁₋₄alkoxy-methyl, C₁₋₄alkyl-thiomethyl, hydroxyethyl, hydroxypropyl, phenyl, aralkyl, C₂₋₄alkenyl or -alkynyl;
R₆ₐ is H or C₁₋₄alkyl;
R₇ₐ is H, C₁₋₄alkyl or a residue of formula (a) as defined above,
Xₐ is O, S, SO or SO₂; and
nₐ is an integer of 1 to 4;
   or a pharmaceutically acceptable salt thereof.

With regard to the compounds of formulae (I) and (II), the term “halogen” encompasses fluorine, chlorine, bromine and iodine. The term “trihalomethyl group” encompasses trifluoromethyl and trichloromethyl. “C₁₋₇ alkyl” encompasses straight-chained or branched alkyl, e.g. methyl, ethyl, propyl, isopropyl, butyl, t-butyl, pentyl, hexyl or heptyl. The phrase “substituted or unsubstituted phenoxy group” encompasses those that have, at any position of its benzene ring, a halogen atom, such as fluorine, chlorine, bromine and iodine, trifluoromethyl, C₁₋₄alkyl or C₁₋₄alkoxy. The term “aralkyl group” as in “aralkyl group” or “aralkyloxy group” encompasses benzyl, diphenylmethyl, phenethyl and phenylpropyl. Any alkyl moiety as present in “C₁₋₄alkoxy”, “C₁₋₄alkylthio”, “C₁₋₄alkylsulfinyl” or “C₁₋₄alkylsulfonyl encompasses straight-chained or branched C₁₋₄alkyl, e.g. methyl, ethyl, propyl, isopropyl or butyl. The phrase “substituted or unsubstituted aralkyl group” encompasses those that have, at any position of its benzene ring, a halogen atom, such as fluorine, chlorine, bromine and iodine, trifluoromethyl, lower alkyl having 1-4 carbon atoms, or lower alkoxy having 1-4 carbon atoms.

Preferred compounds of formula I are compounds of formula Ia

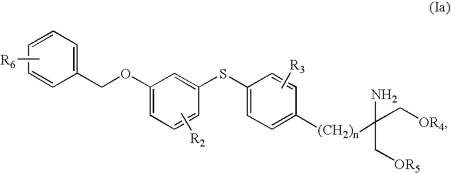

wherein
R₂, R₃, R₄, R₅ and n are as defined above; and
R₆ is hydrogen, halogen, C₁₋₇alkyl, C₁₋₄alkoxy or trifluoromethyl.

Further preferred compounds of formula (Ia) are those wherein R₃ is chlorine, e.g., 2-amino-2-[4-(3-benzyloxyphenylthio)-2-chlorophenyl]ethyl-propane-1,3-diol and its corresponding phosphate derivative, phosphoric acid mono-2-amino-2-[4-(3-benzyloxyphenylthio)-2-chlorophenyl]ethyl-propyl]ester. The phosphoric acid mono-2-amino-2-[4-(3-benzyloxyphenylthio)-2-chlorophenyl]ethyl-propyl]ester can be prepared enantiomerically pure by the procedures described in WO 2005/021503.

Preferred compounds of formula II are compounds of formula (IIa)

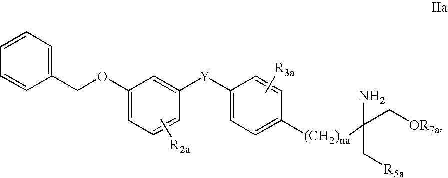

wherein
Y is C or S; and
R₂ₐ, R₃ₐ, R₅ₐ, R₇ₐ and nₐ are as defined above.

Preferred compounds of formula (IIa) are those wherein R₃ is chlorine, e.g., 2-amino-4-[4-(3-benzyloxyphenylthio)-2-chlorophenyl]-2-methylbutane-1-ol; the corresponding phosphoric acid mono-2-amino-4-[4-(3-benzyloxyphenylthio)-2-chlorophenyl]-2-methylbutyl]ester; 2-amino-4-[4-(3-benzyloxyphenylthio)-2-chlorophenyl]-2-ethylbutane-1-ol; and the corresponding phosphoric acid mono-2-amino-4-[4-(3-benzyloxyphenylthio)-2-chlorophenyl]-2-ethylbutyl]ester.

Compounds of formulae I and II are known and are disclosed e.g. in WO03/029205, WO 03/029184 and WO04/026817, respectively, the phosphorylated derivatives being disclosed e.g. in WO04/074297, the contents of which being incorporated herein by reference in their entirety. Compounds of formulae I and II may be prepared as disclosed in above cited references.

Phosphorylated derivatives of compounds of formula (I), e.g., phosphoric acid mono-2-amino-2-[4-(3-benzyloxyphenylthio)-2-chlorophenyl]ethyl-propyl]ester, can be prepared utilizing the procedures for synthesizing phosphorylated compounds described e.g., in WO 2005/021503 (see, e.g., pages 11 and 12). Optically active compounds of structural formula (I) and phosphorylated derivatives thereof, in particular of formula (Ia) can be prepared in high purity utilizing the procedure described, e.g., in Hinterding et al., Synthesis, Vol. 11, pp. 1667-1670 (2003). As an example, an optically active compound of structural formula (Ia), phosphoric acid mono-2-amino-2-[4-(3-benzyloxyphenylthio)-2-chlorophenyl]ethyl-propyl]ester, can be prepared as described in the scheme below utilizing the procedures of Hinterding et al. (2003) supra.

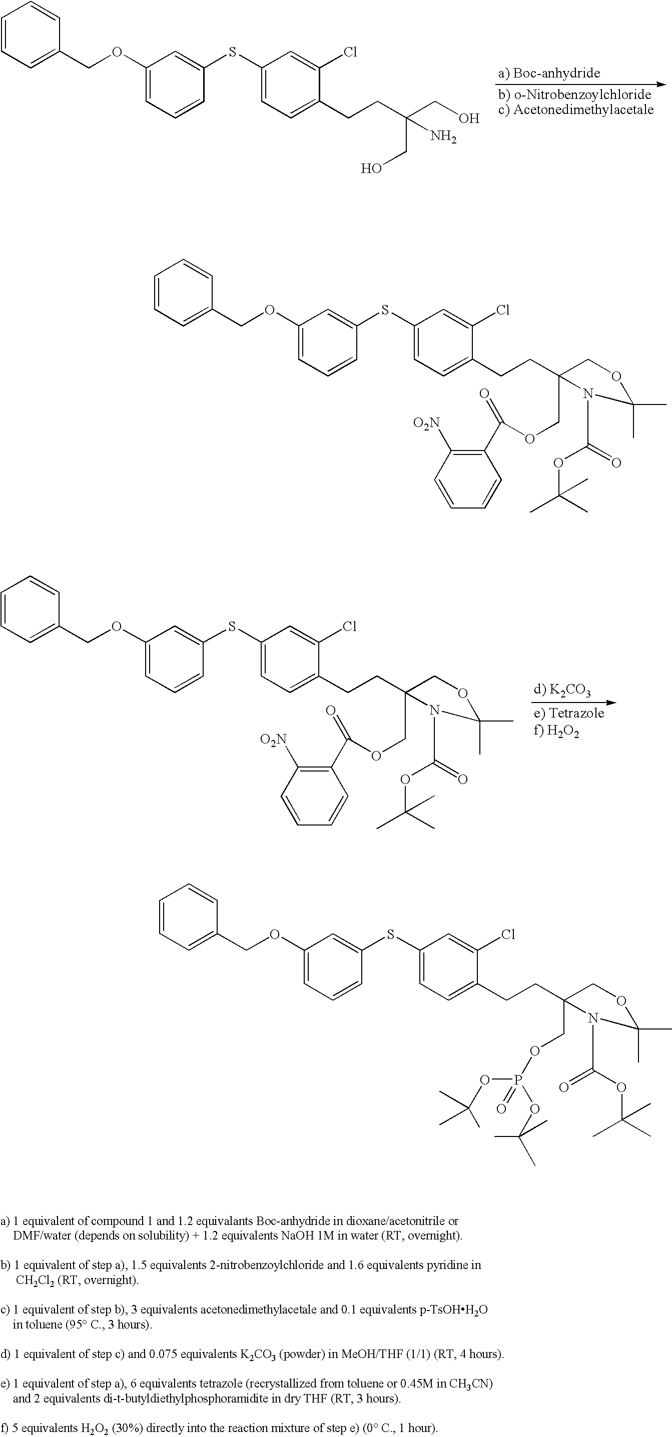

Isolation: the reaction mixture is quenched with sodium thiosulfate (saturated in water) and extracted with ethyl acetate (3×).

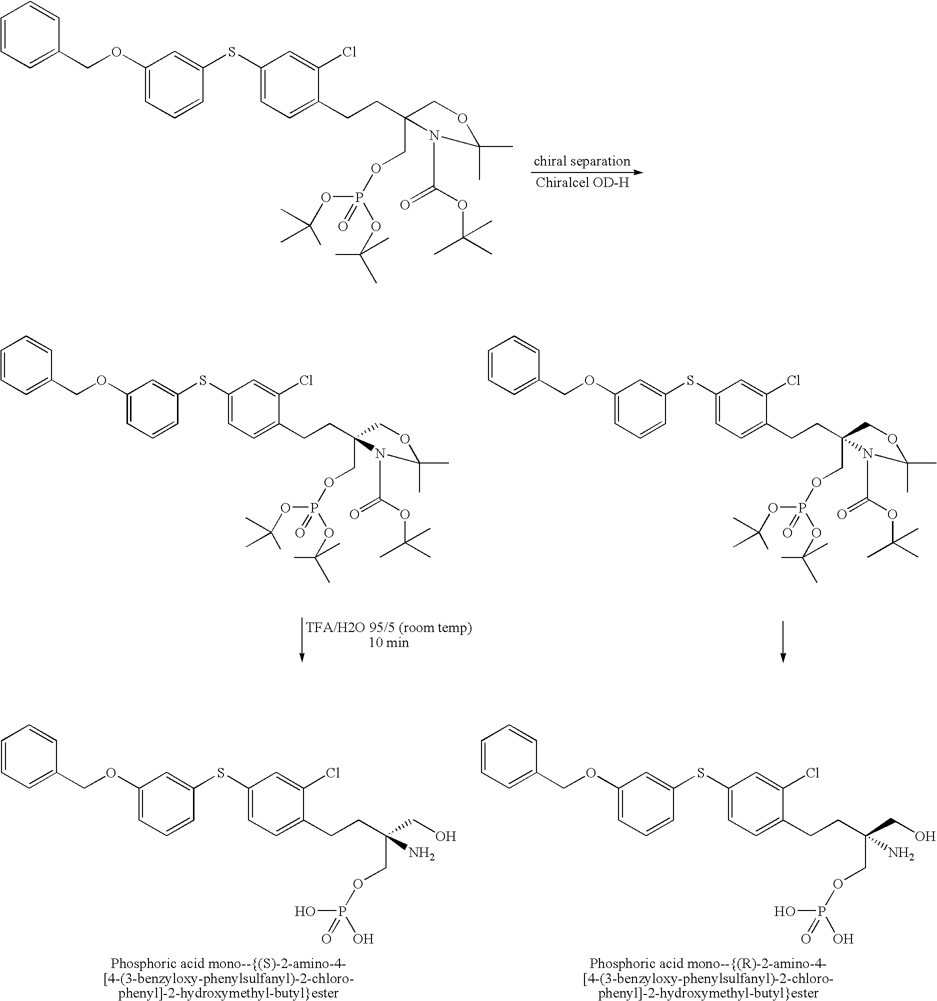

The compounds of formulae II and IIa, e.g., 2-amino-4-[4-(3-benzyloxyphenylthio)-2-chlorophenyl]-2-methylbutane-1-ol and 2-amino-4-[4-(3-benzyloxyphenylthio)-2-chlorophenyl]-2-ethylbutane-1-ol can be prepared as described e.g., in EP 1 548 003 A1. Preparation of such compounds of formulae II and IIa in high optical purity, can be prepared by the procedures described e.g., in Hinterding et al. (2003), supra; and Hinterding et al., Tetra Lett, Vol. 43, No. 45, pp. 8095-8097 (2002). Optically active phosphate derivatives of compounds of structural formulae II and IIa, e.g., phosphoric acid mono-2-amino-4-[4-(3-benzyloxyphenylthio)-2-chlorophenyl]-2-methylbutyl]ester and phosphoric acid mono-2-amino-4-[4-(3-benzyloxyphenylthio)-2-chlorophenyl]-2-ethylbutyl]ester can be prepared in high purity as described in Hinterding et al. (2003), supra.

The compounds of formulae I and II may exist in free or salt form, or as a hydrate. Examples of pharmaceutically acceptable salts of the compounds of the formulae I and II include salts with inorganic acids, such as hydrochloride and hydrobromide, salts with organic acids, such as acetate, trifluoroacetate, citrate, tartrate, methanesulfonate salts.

When the compounds of formulae I and II have one or more asymmetric centers in the molecule, such compounds are to be understood as embracing the various optical isomers, as well as racemates, diastereoisomers and mixtures thereof.

In accordance with the particular findings of the present invention, there is provided

1.1 A method for treating an autoimmune disease selected from the group consisting of peripheral neuritis, optical neuritis, amyotrophic lateral sclerosis and uveitis in a subject in need of such treatment, which method comprises administering to the subject an effective amount of a compound of formula I or II or a pharmaceutically acceptable salt thereof.

1.2 A method for treating multiple sclerosis in a subject in need of such treatment, which method comprises administering to the subject an effective amount of a compound of formula I wherein each of R₄ and R₅ is H or a compound of formula II wherein R₇ₐ is H or C₁₋₄alkyl, or a pharmaceutically acceptable salt thereof.

1.3 A method for alleviating or delaying progression of the symptoms of a demyelinating disease, e.g. multiple sclerosis or Guillain-Barré syndrome, in a subject in need of such treatment, which method comprises administering to the subject an effective amount of a compound of formula I wherein each of R₄ and R₅ is H or a compound of formula II wherein R₇ₐ is H or C₁₋₄alkyl, or a pharmaceutically acceptable salt thereof.

1.4 A method for slowing the progression of physical disability or reducing the rate of clinical relapses in a subject with established multiple sclerosis, which method comprises administering to the subject an effective amount of a compound of formula I wherein each of R₄ and R₅ is H or a compound of formula II wherein R₇ₐ is H or C₁₋₄alkyl, or a pharmaceutically acceptable salt thereof.

1.5 A method for reducing the development of brain lesions or the progression of central nervous system demyelination in a subject with suspected or established multiple sclerosis, which method comprises administering to the subject an effective amount of a compound of formula I wherein each of R₄ and R₅ is H or a compound of formula II wherein R₇ₐ is H or C₁₋₄alkyl, or a pharmaceutically acceptable salt thereof.

1.6 A method for preventing or delaying a second demyelinating event, e.g. a second attack of multiple sclerosis, in a subject in need thereof, which method comprises administering to the subject an effective amount of a compound of formula I wherein each of R₄ and R₅ is H or a compound of formula II wherein R₇ₐ is H or C₁₋₄alkyl, or a pharmaceutically acceptable salt thereof.

1.7 A method for treating optic neuritis in a subject in need thereof, which method comprises administering to the subject an effective amount of a compound of formula I wherein each of R₄ and R₅ is H or a compound of formula II wherein R₇ₐ is H or C₁₋₄alkyl, or a pharmaceutically acceptable salt thereof.

Optic neuritis may be a first symptom associated with a high risk of clinically definite multiple sclerosis.

2.1 A compound of formula I or II or a pharmaceutically acceptable salt thereof, for use in a method according to 1.1 above.

2.2 A compound of formula I wherein each of R₄ and R₅ is H or a compound of formula II wherein R₇ₐ is H or C₁₋₄alkyl, or a pharmaceutically acceptable salt thereof, for use in any one of the methods according to 1.2 to 1.7 above.

3.1 A pharmaceutical composition for use in a method according to 1.1 above, comprising a compound of formula I or II or a pharmaceutically acceptable salt thereof, together with one or more pharmaceutically acceptable diluents or carriers therefor.

3.2 A pharmaceutical composition for use in any one of the methods according to 1.2 to 1.7 above, comprising a compound of formula I wherein each of R₄ and R₅ is H or a compound of formula II wherein R₇ₐ is H or C₁₋₄alkyl, or a pharmaceutically acceptable salt thereof, together with one or more pharmaceutically acceptable diluents or carriers therefor.

4.1 Use of a compound of formula I or II or a pharmaceutically acceptable salt thereof in the preparation of a medicament for use in a method according to 1.1 above.

4.2 Use of a compound of formula I wherein each of R₄ and R₅ is H or a compound of formula II wherein R₇ₐ is H or C₁₋₄alkyl, or a pharmaceutically acceptable salt thereof, in the preparation of a medicament for use in any one of the methods according to 1.2 to 1.7 above.

The term “effective amount” refers to an amount of a compound of formula I or II which, when administered to the patient, is effective to treat an autoimmune disease, such as multiple sclerosis, peripheral neuritis, optical neuritis, amyotrophic lateral sclerosis (Lou Gehrig's disease) and uveitis. With respect to treatment of an autoimmune disease this includes a reduction of symptoms of the disease, and any other indicators known in the art which show the treatment of the autoimmune disease.

Utility of the compounds of formulae I and II in treating the diseases, disorders or conditions as hereinabove specified, may be demonstrated in animal test methods as well as in clinic, for example in accordance with the methods hereinafter described. The most widely used animal model for multiple sclerosis is experimental autoimmune encephalomyelitis (EAE), based on shared histopathological and clinical features with the human disease.

### Methods

Animal models: The monophasic model of acute experimental autoimmune encephalomyelitis (EAE) and the chronic relapsing form are considered to be instructive animal models for multiple sclerosis. EAE can be induced in susceptible animals by a single injection of CNS tissue or MBP emulsified in complete Freund's adjuvant into the base of the tail. A monophasic acute paralytic disease appears in susceptible rat strains, e.g., Lewis, Wistar rat, about 8-11 days post-sensitization. The symptomatic rats recover within the following 7 days, but in other species the attack is usually lethal. In the chronic relapsing disease models rats undergo one to three relapses following the acute disease bout. These relapses are usually from very mild to severe and are observed within 20-100 days after the acute bout.

### 1. Acute EAE Model

Female Lewis rats are immunized by intracutaneous injection in the hind-paws with 0.1 mL of a mixture of guinea pig spinal cord and complete Freund's adjuvant [Difco H37 RA] (3.5 g guinea pig spinal cord+3.5 mL 0.9% NaCl+105 mg M. tuberculosis [Difco H37 RA]+7 mL CFA (Difco H37 RA). Five-ten rats per group are used and somatic symptoms are judged daily on a scale of 0-3. The number of diseased animals as well as the time of onset of the disease is recorded. Test compounds, e.g. a compound of formula I or II, e.g. [Compound A: 2-amino-2-[4-(3-benzyloxyphenylthio)-2-chlorophenyl]ethyl-propane-1,3-diol; Compound B: (R)-2-amino-4-[4-(3-benzyloxyphenylthio)-2-chlorophenyl]-2-methylbutane-1-ol; and Compound C: (R)-2-amino-4-[4-(3-benzyloxyphenylthio)-2-chlorophenyl]-2-ethylbutane-1-ol) are administered daily from days 0-13 days by oral gavage. The statistical significance between treated and untreated groups is analyzed on each day using ANOVA analysis of variance followed by Dunn's multiple comparisons. In the absence of drug treatment symptoms of the disease (paralysis of the tail and both hind legs) usually develop within 8-11 days.

### Clinical Grades:

1=loss of tail tonicity
2=weakness of one or both hind legs, or mild ataxia
3=severe ataxia or paralysis accompanied by urinary incontinence

As shown in Table 1 below, Compounds A, B and C lead to prevention of disease symptoms when administered at doses between 0.1 and 10 mg/kg/day in this model.

TABLE 1

| | Dose | Number of Animals with EAE/total | | | First |
|---|---|---|---|---|---|
| | mg/kg | | Mean score | | symptoms |
| Compound | p.o. | Day 12 | in control | Day 14 | on day |
| Control | — | 5/5 | severity = 2.8 | | 9 |
| Compound A | 0.1 | 0/5 | | 3/4 | 9 |
| | 0.3 | 0/5 | | 1/5 | 14 |
| | 1 | 0/5 | | 0/5 | >14 |
| Control | — | 5/5 | severity = 3 | | 10 |
| Compound B | 1 | 0/5 | | 0/5 | >14 |
| Compound C | 1 | 0/5 | | 0/5 | >14 |

Severity = clinical grades 0-3

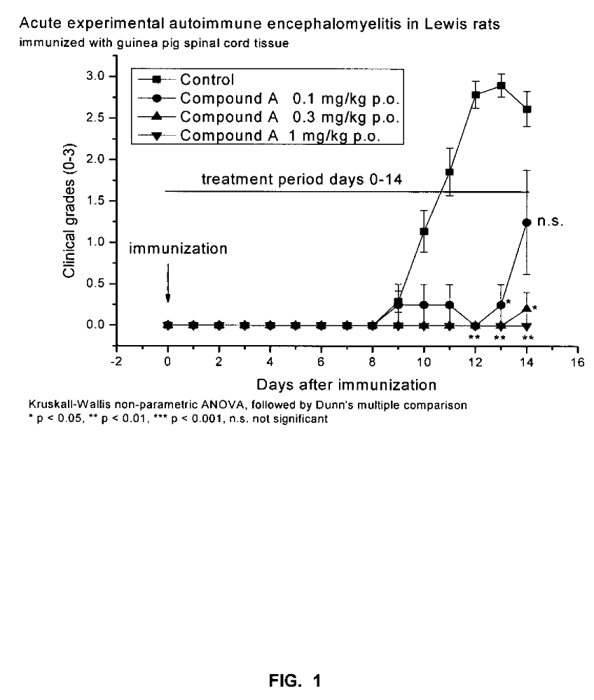

As shown in FIG. 1, Compound A prevents disease symptoms when administered orally at doses between 0.1 and 10 mg/kg/day in this model

FIG. 1 shows the dose response effect of Compound A on prevention of disease symptoms in the acute EAE model.

### 2. Chronic-Relapsing EAE Model

Chronic-relapsing EAE is induced by injecting an emulsion of guinea pig spinal cord in complete Freund's adjuvant in the hind paws of Lewis rats. Six to ten rats per group are used and somatic symptoms are judged daily on a scale of 0-3. The number of diseased animals as well as the time of onset of the disease is recorded. Treatment with the test compound, e.g. a compound of formula I or II, e.g. Compound A as defined supra, is started on day 16 (after first disease bout) and continued until day 31. The statistical significance between treated and untreated groups is analyzed on each day using ANOVA analysis of variance followed by Dunn's multiple comparisons. In the absence of drug treatment 80-100% of the sensitized rats show clinical relapses during the first 40 days following immunization.

### Clinical Grades:

1=loss of tail tonicity
2=weakness of one or both hind legs, or mild ataxia
3=severe ataxia or paralysis accompanied by urinary incontinence

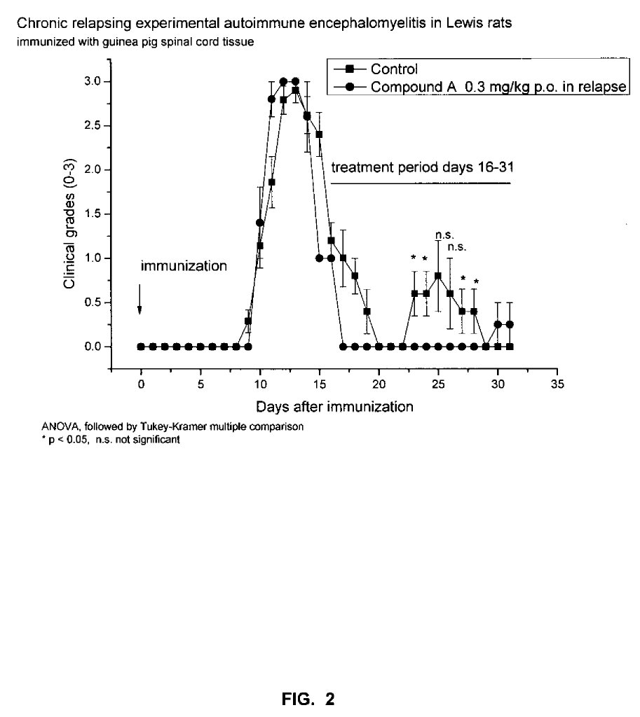

As shown in FIG. 2, Compound A prevents clinical relapses when administered orally at a doses of 0.3 mg/kg/day in the chronic relapsing EAE model. FIG. 2 shows the effect of Compound A on prevention of disease symptoms in the chronic relapsing EAE model.

It is expected that similar results obtained with Compound A would be observed for Compounds B and C.

### 3. Chronic EAE Model

Induction of AEA in DA rat is induced as described by Lorentzen et al, 1995, J. Neuroimmunol.; 63(2):193-205 and Adelmann et al, 1995, J. Neuroimmunol.; 63(1):17-27. Briefly, rats are immunized with a mixture of DA rat brain and DA rat and bovine spinal cord homogenate supplemented with 0.02 μg/ml purified recombinant rat MOG protein. The mixture is homogenized and then mixed 1:1 with complete Freund's adjuvant containing 4 mg/ml M. tuberculosis H37RA(CFA). The resultant mixture is then homogenized using a Polytron PT3100 homogenizer (Kinematica, Lucerne, Switzerland). Rats are then injected subcutaneously at the dorsal root of tail with a single injection of 200 μl antigen/CFA. The resultant chronic disease is evaluated using numeric scale of progressive paralysis: 0, no paralysis; 1, loss of tail tonicity; 2, hindlimb weakening or ataxia; 3, hindlimb paralysis with or without urinary incontinence; 4, hindlimb and forelimb paralysis; 5, moribund or death. Clinical scores are evaluated on a daily basis, while body weight is determined every other day. At the peak of clinical disease, prior to treatment, animal groups are rearranged such that the clinical disease scores are comparable. Treatment of animals begins at the peak clinical disease on the 12^{th} day and continues daily the 33^{rd} day post-immunization (total 22 days). The test compound or vehicle (for the control groups) is administered orally daily.

In this assay, Compound A administered orally at a dose of 0.3, 0.1 or 0.03 mg/kg/d effectively inhibits chronic EAE. Statistically analysis demonstrates significant reduction in clinical disease at each dose of Compound A compared to that of the vehicle group.

### Clinical Trial

Suitable clinical studies are, e.g., open-label, dose-escalation or randomized, double-blind studies in patients with the aforementioned demyelinating diseases, multiple sclerosis, peripheral neuritis, optical neuritis, amyotrophic lateral sclerosis and uveitis. The beneficial effects on these autoimmune diseases, can be determined directly through the results of these studies which are known as such to a person skilled in the art. Such studies may also be suitable to compare the effects of a monotherapy using compounds of formula I or II as active ingredient and a combination of such compounds with a second drug substance.

For example, 50 patients with relapsing-remitting multiple sclerosis receive the test compound, e.g a compound of formula I or II, preferably a compound of formula I wherein each of R₄ and R₅ is H or a compound of formula II wherein R₇ₐ is H or C₁₋₄alkyl, or a pharmaceutically acceptable salt thereof, at a daily dosage of 0.5 to 50 mg p.o. The general clinical state of the patient is investigated weekly by physical and laboratory examination. Disease state and changes in disease progression are assessed every 2 months by radiological examination (MRI) and physical examination. Initially patients receive treatment for 2 to 6 months. Thereafter, they remain on treatment for as long as their disease does not progress and the drug is satisfactorily tolerated.

Main variables for evaluation: Safety (adverse events), standard serum biochemistry and hematology, magnetic resonance imaging (MRI).

Instead of patients with relapsing-remitting multiple sclerosis, patients having a first isolated, well-defined neurologic event consistent with demyelination and e.g. involving the optic nerve (unilateral optic neuritis), spinal cord (e.g. incomplete transverse myelitis) or brain stem or cerebellum (brain-stem or cerebellar syndrome) confirmed on opthalmologic or neurologic examination, may undergo clinical treatment with a compound of formula I or II, preferably a compound of formula I wherein each of R₄ and R₅ is H or a compound of formula II wherein R₇ₐ is H or C₁₋₄alkyl, or a pharmaceutically acceptable salt thereof.

Daily dosages required in practicing the method of the present invention will vary depending upon, for example, the compound used, the host, the mode of administration and the severity of the condition to be treated. A preferred daily dosage range is about from 0.1 to 100 mg as a single dose or in divided doses. Suitable daily dosages for patients are on the order of from e.g. 0.1 to 50 mg p.o. The compound may be administered by any conventional route, in particular enterally, e.g. orally, e.g. in the form of tablets, capsules, drink solutions, nasally, pulmonary (by inhalation) or parenterally, e.g. in the form of injectable solutions or suspensions. Suitable unit dosage forms for oral administration comprise from ca. 0.1 to 30 mg, usually 0.25 to 30 mg active ingredient, e.g. from about 0.1-5 mg, together with one or more pharmaceutically acceptable diluents or carriers therefore.

Compounds of formula I or II may be administered by any conventional route, in particular, enterally, e.g., orally, e.g., in the form of tablets or capsules, or parenterally, e.g., in the form of injectable solutions or suspensions, topically, e.g., in the form of lotions, gels, ointments or creams, or in a nasal or a suppository form. Phosphate derivatives of the compounds of formula I or II are preferably administered parenterally. Pharmaceutical compositions comprising such compounds in free form or in pharmaceutically acceptable salt form in association with at least one pharmaceutical acceptable carrier or diluent may be manufactured in conventional manner by mixing with a pharmaceutically acceptable carrier or diluent.

The compounds of formula I or II may be administered in free form or in pharmaceutically acceptable salt form, e.g., as indicated above. Such salts may be prepared in conventional manner and exhibit the same order of activity as the free compounds.

Compounds of formula I or II may be administered as the sole active ingredient or in conjunction with, e.g., as an adjuvant to, other drugs, e.g., immunosuppressive or immunomodulating agents or other anti-inflammatory agents for the treatment of the afore-mentioned autoimmune disorders. For example, the compounds may be used in combination with interferons, e.g., pegylated or non-pegylated α-interferons, β-interferons or γ-interferons, e.g., administered by subcutaneous, intramuscular or oral routes; an altered peptide ligand, such as Glatiramer, e.g., in the acetate form; monoclonal antibodies to various T-cell surface markers, e.g. natalizumab (ANTEGREN®) or alemtuzumab; an ascomycin having immunosuppressive properties, e.g., ABT-281, ASM981, etc.; a steroid, e.g. methylprednisolone, prednisone or dexamethasone; a corticosteroid; cyclophosphamide; azathioprine; methotrexate; mitoxantrone; leflunomide; mizoribine; mycophenolic acid; mycophenolate mofetil; 15-deoxyspergualine or an immunosuppressive homologue, analogue or derivative thereof; immunosuppressive monoclonal antibodies, e.g., monoclonal antibodies to leukocyte receptors, e.g., MHC, CD2, CD3, CD4, CD7, CD8, CD25, CD28, CD40, CD45, CD58, CD80, CD86 or their ligands; other immunomodulatory compounds, e.g., a recombinant binding molecule having at least a portion of the extracellular domain of CTLA4 or a mutant thereof, e.g., an at least extracellular portion of CTLA4 or a mutant thereof joined to a non-CTLA4 protein sequence, e.g., CTLA41g, e.g., designated ATCC 68629, or a mutant thereof, e.g., LEA29Y; adhesion molecule inhibitors, e.g., LFA-1 antagonists, ICAM-1 or -3 antagonists, VCAM-4 antagonists or VLA-4 antagonists; cathepsin S inhibitors; mTOR inhibitors, e.g., rapamycin, 40-O-(2-hydroxyethyl)-rapamycin, CCI779 or ABT578; calcineurin inhibitors, e.g., cyclosporin A, FK 506 or ISA Tx247.

Where compounds of formula I or III are administered in conjunction with other immunosuppressive/immunomodulatory or anti-inflammatory therapy, dosages of the co-administered immunosuppressant, immunomodulatory or anti-inflammatory compound will of course vary depending on the type of co-drug employed, e.g., whether it is a steroid or a calcineurin inhibitor, on the specific drug employed, on the condition being treated and so forth. Accordingly, in yet a further aspect, the invention provides:

5. A method as defined above comprising co-administration, e.g. concomitantly or in sequence, of a therapeutically effective non-toxic amount of a compound of formula I or II or a pharmaceutically acceptable salt thereof, e.g. a compound of formula I wherein each of R₄ and R₅ is H or a compound of formula II wherein R₇₂ is H or C₁₋₄alkyl, and at least a second drug substance, e.g. as indicated above.

6. A pharmaceutical combination, e.g. a kit, comprising a) a first agent which is a compound of formula I or II or a pharmaceutically acceptable salt thereof, e.g. a compound of formula I wherein each of R₄ and R₅ is H or a compound of formula II wherein R₇ₐ is H or C₁₋₄alkyl, in free form or in pharmaceutically acceptable salt form, and b) at least a second drug substance, e.g. as indicated above. The kit may comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g., a compound of the invention and a second drug substance, are both administered to a patient simultaneously in the form of a single entity or dosage. The term “non-fixed combination” means that the active ingredients, e.g., a compound of the invention and a second drug substance, are both administered to a patient as separate entities either simultaneously, concurrently or sequentially with no specific time limits, wherein such administration provides therapeutically effective levels of the 2 compounds in the body of the patient. The latter also applies to cocktail therapy, e.g., the administration of 3 or more active ingredients.

1. A method for the treatment of an autoimmune disease comprising administering to the subject an effective amount of a compound of formula I 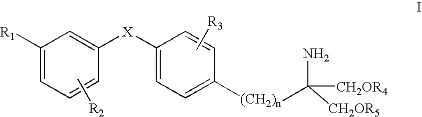
wherein X is O, S, SO or SO₂;
R₁ is halogen, trihalomethyl, OH, C₁₋₇alkyl, C₁₋₄alkoxy, trifluoromethoxy, phenoxy, cyclohexylmethyloxy, pyridylmethoxy, cinnamyloxy, naphthylmethoxy, phenoxymethyl, CH₂—OH, CH₂—CH₂—OH, C₁₋₄alkylthio, C₁₋₄alkylsulfinyl, C₁₋₄alkylsulfonyl, benzylthio, acetyl, nitro or cyano, or phenyl, phenylC₁₋₄alkyl or phenyl-C₁₋₄alkoxy each phenyl group thereof being optionally substituted by halogen, CF₃, C₁₋₄alkyl or C₁₋₄alkoxy;
R₂ is H, halogen, trihalomethyl, C₁₋₄alkoxy, C₁₋₇alkyl, phenethyl or benzyloxy;
R₃ is H, halogen, CF₃, OH, C₁₋₇alkyl, C₁₋₄alkoxy, benzyloxy, phenyl or C₁₋₄alkoxymethyl;
each of R₄ and R₅, independently is H or a residue of formula (a) 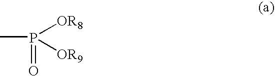
wherein each of R₈ and R₉, independently, is H or C₁₋₄alkyl optionally substituted by halogen;
and n is an integer from 1 to 4;
or a pharmaceutically acceptable salt thereof,
or a compound of formula II 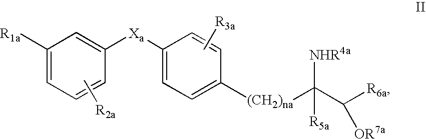
wherein
R₁ₐ is halogen, trihalomethyl, C₁₋₄alkyl, C₁₋₄alkoxy, C₁₋₄alkylthio, C₁₋₄alkylsulifinyl, C₁₋₄alkyl-sulfonyl, aralkyl, optionally substituted phenoxy or aralkyloxy;
R₂ₐ is H, halogen, trihalomethyl, C₁₋₄alkyl, C₁₋₄alkoxy, aralkyl or aralkyloxy;
R₃ₐ is H, halogen, CF₃, C₁₋₄alkyl, C₁₋₄alkoxy, C₁₋₄alkylthio or benzyloxy;
R₄ₐ is H, C₁₋₄alkyl, phenyl, optionally substituted benzyl or benzoyl, or lower aliphatic C₁₋₅acyl;
R₅ₐ is H, monohalomethyl, C₁₋₄alkyl, C₁₋₄alkoxy-methyl, C₁₋₄alkyl-thiomethyl, hydroxyethyl, hydroxypropyl, phenyl, aralkyl, C₂₋₄alkenyl or -alkynyl;
R₆ₐ is H or C₁₋₄alkyl;
R₇ₐ is H, C₁₋₄alkyl or a residue of formula (a) as defined above,
Xₐ is O, S, SO or SO₂; and
nₐ is an integer of 1 to 4;
or a pharmaceutically acceptable salt thereof.

2. (canceled)

3. A method for alleviating or delaying progression of the symptoms of a demyelinating disease comprising administering to the subject an effective amount a compound of formula I 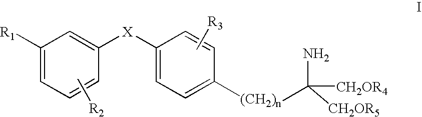
wherein X is O, S, SO or SO₂;
R₁ is halogen, trihalomethyl, OH, C₁₋₇alkyl, C₁₋₄alkoxy, trifluoromethoxy, phenoxy, cyclohexylmethyloxy, pyridylmethoxy, cinnamyloxy, naphthylmethoxy, phenoxymethyl, CH₂—OH, CH₂—CH₂—OH, C₁₋₄alkylthio, C₁₋₄alkylsulfinyl, C₁₋₄alkylsulfonyl, benzylthio, acetyl, nitro or cyano, or phenyl, phenylC₁₋₄alkyl or phenyl-C₁₋₄alkoxy each phenyl group thereof being optionally substituted by halogen, CF₃, C₁₋₄alkyl or C₁₋₄alkoxy;
R₂ is H, halogen, trihalomethyl, C₁₋₄alkoxy, C₁₋₇alkyl, phenethyl or benzyloxy;
R₃ is H, halogen, CF₃, OH, C₁₋₄alkyl, C₁₋₄alkoxy, benzyloxy, phenyl or C₁₋₄alkoxymethyl;
each of R₄ and R₅, independently is H; and
and n is an integer from 1 to 4;
or a pharmaceutically acceptable salt thereof,
or a compound of formula II 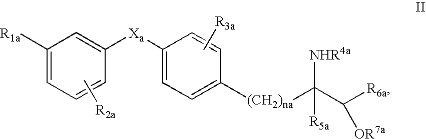
wherein
R₁ₐ is halogen, trihalomethyl, C₁₋₄alkyl, C₁₋₄alkoxy, C₁₋₄alkylthio, C₁₋₄alkylsulifinyl, C₁₋₄alkyl-sulfonyl, aralkyl, optionally substituted phenoxy or aralkyloxy;
R₂ₐ is H, halogen, trihalomethyl, C₁₋₄alkyl, C₁₋₄alkoxy, aralkyl or aralkyloxy;
R₃ₐ is H, halogen, CF₃, C₁₋₄alkyl, C₁₋₄alkoxy, C₁₋₄alkylthio or benzyloxy;
R₄ₐ is H, C₁₋₄alkyl, phenyl, optionally substituted benzyl or benzoyl, or lower aliphatic C₁₋₅acyl;
R₅ₐ is H, monohalomethyl, C₁₋₄alkyl, C₁₋₄alkoxy-methyl, C₁₋₄alkyl-thiomethyl, hydroxyethyl, hydroxypropyl, phenyl, aralkyl, C₂₋₄alkenyl or -alkynyl;
R₆ₐ is H or C₁₋₄alkyl;
R₇ₐ is H or C₁₋₄alkyl,
Xₐ is O, S, SO or SO₂; and
nₐ is an integer of 1 to 4;
or a pharmaceutically acceptable salt thereof.

4. A method of slowing the progression of physical disability or reducing the rate of clinical relapses in a subject with established multiple sclerosis comprising administering to the subject an effective amount of a compound of formula I 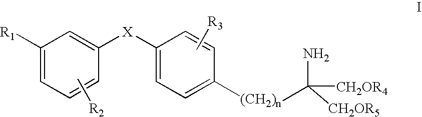
wherein X is O, S, SO or SO₂;
R₁ is halogen, trihalomethyl, OH, C₁₋₄alkyl, C₁₋₄alkoxy, trifluoromethoxy, phenoxy, cyclohexylmethyloxy, pyridylmethoxy, cinnamyloxy, naphthylmethoxy, phenoxymethyl, CH₂—OH, CH₂—CH₂—OH, C₁₋₄alkylthio, C₁₋₄alkylsulfinyl, C₁₋₄alkylsulfonyl, benzylthio, acetyl, nitro or cyano, or phenyl, phenylC₁₋₄alkyl or phenyl-C₁₋₄alkoxy each phenyl group thereof being optionally substituted by halogen, CF₃, C₁₋₄alkyl or C₁₋₄alkoxy;
R₂ is H, halogen, trihalomethyl, C₁₋₄alkoxy, C₁₋₇alkyl, phenethyl or benzyloxy;
R₃ is H, halogen, CF₃, OH, C₁₋₇alkyl, C₁₋₄alkoxy, benzyloxy, phenyl or C₁₋₄alkoxymethyl;
each of R₄ and R₅, independently is H; and
and n is an integer from 1 to 4;
or a pharmaceutically acceptable salt thereof,
or a compound of formula II 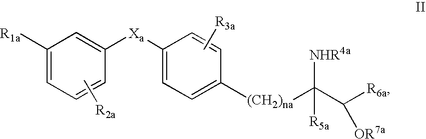
wherein
R₁ₐ is halogen, trihalomethyl, C₁₋₄alkyl, C₁₋₄alkoxy, C₁₋₄alkylthio, C₁₋₄alkylsulifinyl, C₁₋₄alkyl-sulfonyl, aralkyl, optionally substituted phenoxy or aralkyloxy;
R₂ₐ is H, halogen, trihalomethyl, C₁₋₄alkyl, C₁₋₄alkoxy, aralkyl or aralkyloxy;
R₃ₐ is H, halogen, CF₃, C₁₋₄alkyl, C₁₋₄alkoxy, C₁₋₄alkylthio or benzyloxy;
R₄ₐ is H, C₁₋₄alkyl, phenyl, optionally substituted benzyl or benzoyl, or lower aliphatic C₁₋₅acyl;
R₅ₐ is H, monohalomethyl, C₁₋₄alkyl, C₁₋₄alkoxy-methyl, C₁₋₄alkyl-thiomethyl, hydroxyethyl, hydroxypropyl, phenyl, aralkyl, C₂₋₄alkenyl or -alkynyl;
R₆ₐ is H or C₁₋₄alkyl;
R₇ₐ is H or C₁₋₄alkyl,
Xₐ is O, S, SO or SO₂; and
nₐ is an integer of 1 to 4;
or a pharmaceutically acceptable salt thereof.

5. A method for reducing the development of brain lesions or the progression of central nervous system demyelination in a subject with suspected or established multiple sclerosis comprising administering to the subject an effective amount of a compound of formula I 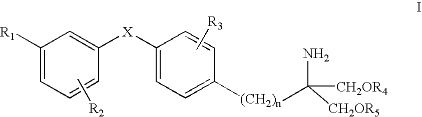
wherein X is O, S, SO or SO₂;
R₁ is halogen, trihalomethyl, OH, C₁₋₇alkyl, C₁₋₄alkoxy, trifluoromethoxy, phenoxy, cyclohexylmethyloxy, pyridylmethoxy, cinnamyloxy, naphthylmethoxy, phenoxymethyl, CH₂—OH, CH₂—CH₂—OH, C₁₋₄alkylthio, C₁₋₄alkylsulfinyl, C₁₋₄alkylsulfonyl, benzylthio, acetyl, nitro or cyano, or phenyl, phenylC₁₋₄alkyl or phenyl-C₁₋₄alkoxy each phenyl group thereof being optionally substituted by halogen, CF₃, C₁₋₄alkyl or C₁₋₄alkoxy;
R₂ is H, halogen, trihalomethyl, C₁₋₄alkoxy, C₁₋₇alkyl, phenethyl or benzyloxy;
R₃ is H, halogen, CF₃, OH, C₁₋₇alkyl, C₁₋₄alkoxy, benzyloxy, phenyl or C₁₋₄alkoxymethyl;
each of R₄ and R₅, independently is H; and
and n is an integer from 1 to 4;
or a pharmaceutically acceptable salt thereof,
or a compound of formula II 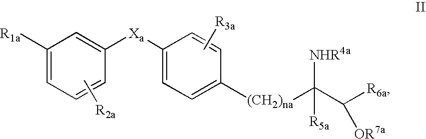
wherein
R₁ₐ is halogen, trihalomethyl, C₁₋₄alkyl, C₁₋₄alkoxy, C₁₋₄alkylthio, C₁₋₄alkylsulifinyl, C₁₋₄alkyl-sulfonyl, aralkyl, optionally substituted phenoxy or aralkyloxy;
R₂ₐ is H, halogen, trihalomethyl, C₁₋₄alkyl, C₁₋₄alkoxy, aralkyl or aralkyloxy;
R₃ₐ is H, halogen, CF₃, C₁₋₄alkyl, C₁₋₄alkoxy, C₁₋₄alkylthio or benzyloxy;
R₄ₐ is H, C₁₋₄alkyl, phenyl, optionally substituted benzyl or benzoyl, or lower aliphatic C₁₋₅acyl;
R₅ₐ is H, monohalomethyl, C₁₋₄alkyl, C₁₋₄alkoxy-methyl, C₁₋₄alkyl-thiomethyl, hydroxyethyl, hydroxypropyl, phenyl, aralkyl, C₂₋₄alkenyl or -alkynyl;
R₆ₐ is H or C₁₋₄alkyl;
R₇ₐ is H or C₁₋₄alkyl,
Xₐ is O, S, SO or SO₂; and
nₐ is an integer of 1 to 4;
or a pharmaceutically acceptable salt thereof.

6. A method for preventing or delaying a second demyelinating event comprising administering to the subject an effective amount of a compound of formula I 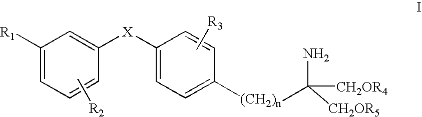
wherein X is O, S, SO or SO₂;
R₁ is halogen, trihalomethyl, OH, C₁₋₇alkyl, C₁₋₄alkoxy, trifluoromethoxy, phenoxy, cyclohexylmethyloxy, pyridylmethoxy, cinnamyloxy, naphthylmethoxy, phenoxymethyl, CH₂—OH, CH₂—CH₂—OH, C₁₋₄alkylthio, C₁₋₄alkylsulfinyl, C₁₋₄alkylsulfonyl, benzylthio, acetyl, nitro or cyano, or phenyl, phenylC₁₋₄alkyl or phenyl-C₁₋₄alkoxy each phenyl group thereof being optionally substituted by halogen, CF₃, C₁₋₄alkyl or C₁₋₄alkoxy;
R₂ is H, halogen, trihalomethyl, C₁₋₄alkoxy, C₁₋₇alkyl, phenethyl or benzyloxy;
R₃H, halogen, CF₃, OH, C₁₋₇alkyl, C₁₋₄alkoxy, benzyloxy, phenyl or C₁₋₄alkoxymethyl;
each of R₄ and R₅, independently is H; and
and n is an integer from 1 to 4;
or a pharmaceutically acceptable salt thereof,
or a compound of formula II 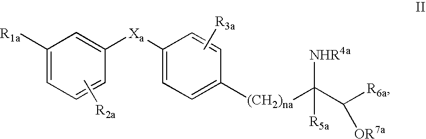
wherein
R₁ₐ is halogen, trihalomethyl, C₁₋₄alkyl, C₁₋₄alkoxy, C₁₋₄alkylthio, C₁₋₄alkylsulifinyl, C₁₋₄alkyl-sulfonyl, aralkyl, optionally substituted phenoxy or aralkyloxy;
R₂ₐ is H, halogen, trihalomethyl, C₁₋₄alkyl, C₁₋₄alkoxy, aralkyl or aralkyloxy;
R₃ₐ is H, halogen, CF₃, C₁₋₄alkyl, C₁₋₄alkoxy, C₁₋₄alkylthio or benzyloxy;
R₄ₐ is H, C₁₋₄alkyl, phenyl, optionally substituted benzyl or benzoyl, or lower aliphatic C₁₋₅acyl;
R₅ₐ is H, monohalomethyl, C₁₋₄alkyl, C₁₋₄alkoxy-methyl, C₁₋₄alkyl-thiomethyl, hydroxyethyl, hydroxypropyl, phenyl, aralkyl, C₂₋₄alkenyl or -alkynyl;
R₆ₐ is H or C₁₋₄alkyl;
R₇ₐ is H or C₁₋₄alkyl,
Xₐ is O, S, SO or SO₂; and
nₐ is an integer of 1 to 4;
or a pharmaceutically acceptable salt thereof.

7-9. (canceled)

10. A pharmaceutical composition for use in the treatment of peripheral neuritis, optic neuritis, amyotrophic lateral sclerosis or uveitis, comprising a compound of formula I or II as defined in claim 1 or a pharmaceutically acceptable salt thereof, together with one or more pharmaceutically acceptable diluents or carriers therefor.

11. The method of claim 1 wherein the autoimmune disease is selected from the group consisting of peripheral neuritis, optic neuritis, amyotrophic lateral sclerosis and uveitis.

12. (canceled)

13. A method according to claim 1, comprising co-administration to the subject, concomitantly or in sequence, of at least a second drug.

14. The composition according to claim 10, further comprising at least a second drug.

15. The method according to claim 1 wherein the compound of Formula I comprises a compound of Formula Ia 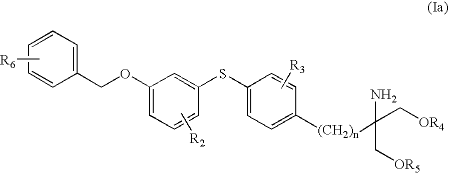
wherein
R₁ is halogen, trihalomethyl, OH, C₁₋₇alkyl, C₁₋₄alkoxy, trifluoromethoxy, phenoxy, cyclohexylmethyloxy, pyridylmethoxy, cinnamyloxy, naphthylmethoxy, phenoxymethyl, CH₂—OH, CH₂—CH₂—OH, C₁₋₄alkylthio, C₁₋₄alkylsulfinyl, C₁₋₄alkylsulfonyl, benzylthio, acetyl, nitro or cyano, or phenyl, phenylC₁₋₄alkyl or phenyl-C₁₋₄alkoxy each phenyl group thereof being optionally substituted by halogen, CF₃, C₁₋₄alkyl or C₁₋₄alkoxy;
R₂ is H, halogen, trihalomethyl, C₁₋₄alkoxy, C₁₋₇alkyl, phenethyl or benzyloxy;
R₃ is H, halogen, CF₃, OH, C₁₋₇alkyl, C₁₋₄alkoxy, benzyloxy, phenyl or C₁₋₄alkoxymethyl;
each of R₄ and R₅, independently is H;
R₆ is H, halogen, C₁₋₇alkyl, C₁₋₄alkoxy or trifluoromethyl; and
n is an integer of 1 to 4;
or a pharmaceutically acceptable salt thereof.

16. The method according to claim 15 wherein the compound of Formula Ia is selected from the group consisting of 2-amino-2-[4-(3-benzyloxyphenylthio)-2-chlorophenyl]ethyl-propane-1,3-diol and phosphoric acid mono-2-amino-2-[4-(3˜benzyloxyphenylthio)-2-chlorophenyl]ethyl-propyl]ester.

17. The method according to claim 1 wherein the compound of Formula II comprises a compound of Formula IIa 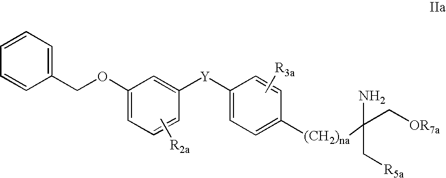
wherein Y is O or S; and
R₂ₐ is H, halogen, trihalomethyl, C₁₋₄alkyl, C₁₋₄alkoxy, aralkyl or aralkyloxy;
R₃ₐ is H, halogen, CF₃, C₁₋₄alkyl, C₁₋₄alkoxy, C₁₋₄alkylthio or benzyloxy;
R₅ₐ is H, monohalomethyl, C₁₋₄alkyl, C₁₋₄alkoxy-methyl, C₁₋₄alkyl-thiomethyl, hydroxyethyl, hydroxypropyl, phenyl, aralkyl, C₂₋₄alkenyl or -alkynyl;
R₇ₐ is H or C₁₋₄alkyl; and
nₐ is an integer of 1 to 4;
or a pharmaceutically acceptable salt thereof.

18. The method according to claim 17 wherein the compound of Formula IIa is selected from the group consisting of 2-amino-4-[4-(3-benzyloxyphenylthio)-2-chlorophenyl]-2-methylbutane-1-ol, phosphoric acid mono-2-amino-4-[4-(3-benzyloxyphenylthio)-2-chlorophenyl]-2-methylbutyl]ester, 2-amino-4-[4-(3-benzyloxyphenylthio)-2-chlorophenyl]-2-ethylbutane-1-ol, and phosphoric acid mono-2-amino-4-[4-(3-benzyloxyphenylthio)-2-chlorophenyl]-2-ethylbutyl]ester.

19. The method according to claim 1 wherein R₄ and R₅ of Formula I independently are H or R₇ₐ of Formula II is H or C₁₋₄alkyl.

20. The method according to claim 19 wherein the autoimmune disease is multiple sclerosis.

21. A pharmaceutical composition for use in the treatment of multiple sclerosis, comprising a compound of formula I or II as defined in claim 19 or a pharmaceutically acceptable salt thereof, together with one or more pharmaceutically acceptable diluents or carriers therefor.
